(19) United States
(12) Patent Application Publication
(10) Pub. No.: US 2008/0069864 A1
(43) Pub. Date: Mar. 20, 2008

(54) Delivery System for Cosmetic and Skincare Products

(75) Inventors: Craig J. Berry, Westport, CT (US); Jill O'Neill, Matamoras, PA (US)

Correspondence Address: PERMAN & GREEN, 425 POST ROAD, FAIRFIELD, CT, 06824, US

(73) Assignee: ACUPAC Packaging, Inc., Mahwah, NJ (US)

(21) Appl. No.: 11/865,175

(22) Filed: Oct. 1, 2007

Related U.S. Application Data
(62) Division of application No. 10/790,351, filed on Mar. 1, 2004.
(60) Provisional application No. 60/450,956, filed on Feb. 28, 2003.

(51) Int. Cl. A61K 47/00; A61L 15/16; A61P 17/00

(52) U.S. Cl. 424/444; 514/777; 514/779; 514/781; 514/789

(57) ABSTRACT

Cosmetic and skin care products are dispersed or encapsulated within a polymer matrix to provide a convenient means of storing and transporting the cosmetic or skin care product prior to use. Prior to use the polymer matrix is solubilized or melted to release the cosmetic or skin care product to the user's skin.

This application is a divisional of, and claims priority to, U.S. application Ser. No. 10/790,351, filed on Mar. 1, 2004, which claims benefit to U.S. Provisional Application Ser. No. 60/450,956, filed on Feb. 28, 2003.

### BACKGROUND OF THE INVENTION

The art of cosmetics has advanced significantly since prehistoric times. While the purpose is largely still enhancement, the trend is toward a more natural look, and emphasizing good features with more subtle strategies. To this end, a considerable focus has been devoted to development of cosmetics that subtly persuade the observer's eye to see a more perfect image of the face, e.g., one having no lines or blemishes, than actually exists, without the use of large quantities of heavy opaque pigments or colorants to cover flaws. There has also been a strong focus on maintaining a youthful and healthy skin and to prevent premature aging or degradation of the skin structure.

Thus many present cosmetics have activities to inhibit the skin aging and to improve the skin, in addition to the moisturizing function. Aging-preventing materials employed in cosmetics are broadly classified into peroxide lipid inhibitors and cell activators.

There are a number of skin care products on the market in the form of creams, ointments, lotions, gels or emulsions. The majority of these products is not miscible with water and generally contains numerous ingredients (e.g., about 20 to 40 different ingredients). One problem with these complex facial skin care products is that they are relatively difficult to prepare, often requiring special skills and facilities. Additionally, due to their numerous ingredients and methods of preparation, the products are often very expensive.

Current facial skin care products are also relatively unstable in chemical composition due to interaction of the multiple ingredients in the composition. There exists a need for skin care methods and formulations that are inexpensive, easy to prepare, and highly stable, as well as highly effective in producing facial cosmetic benefits or improvements.

Furthermore, many of the current products are heavy, bulky or otherwise unwieldy or uncomfortable to carry. This prevents the user from having access to the product when and where it may be needed as when skin is exposed to an adverse environment, such as windy conditions, and dries out or would otherwise desirably need conditioning.

Thus, there exists a need for an easy to carry, easy to use, lightweight, cosmetic or skin care product where the container causes no disposal problems.

### SUMMARY OF THE INVENTION

Disclosed is a cosmetic or skin care product that is neither a cream, ointment, lotion, gel nor emulsion but is rather a solid product where the active ingredients are dispersed or encapsulated in a solid film which disappears when the product is applied to the skin of the user.

The solid film containing the active ingredients is either a liquid soluble, preferably a water soluble, thin film or a thermally degradable thin film, in either case with or without multiple layers.

A significant advantage is that the thin film is dispensed in unit dose sizes thus allowing the user to apply an appropriate amount of product each time.

The thin films may be shaped in an infinite number of shapes, to provide an attractive and convenient shape for each use. Thin films intended for different uses, such as for dry or oily skin, may be shaped differently thus providing a quick method of selecting the appropriate product.

Products intended for different uses or simply for attractiveness of presentation may be colored differently.

### DETAILED DESCRIPTION OF THE PREFERRED EMBODIMENT(s)

The disclosed product comprises a film forming, liquid, preferably water, soluble polymer and a cosmetic or skin care product incorporated within the polymer matrix.

Where the film forming, water soluble polymer is water soluble it may be composed of, but is not limited to polymers selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, pectin, tragacanth gum, guar gum, acacia gum, Arabic gum, polyacrylic acid, methylmethacrylate copolymer, carboxyvinyl polymer, gelatin, amylase, high amylase starch, hydroxypropylated high amylase starch, dextrin, chitin, chitosan, levan, elsinan, collagen, zein, gluten, soy protein isolate, whey protein isolate, casein and mixtures thereof.

Preferred film forming water soluble polymers are pullulan, hydroxypropylethyl cellulose, hydroxyethyl cellulose, hydroxypropyl cellulose, carboxymethyl cellulose, sodium alginate, pectin, and mixtures thereof.

The most preferred film forming water-soluble polymers are pullulan, pectin, and mixtures thereof.

Other ingredients may be added to the blend for the purpose of stabilizing and plasticizing the film.

The film may be manufactured by conventional means and cut into various shapes.

The finished film is preferably packaged in moisture retardant packaging.

Since the liquid to activate the product is supplied by the user at the time of use, the product is lightweight and unmessy in its passive state. The liquid to activate the product and dissolve the film can come, when the activating liquid is water, from surface moisture on the users skin, either naturally occurring moisture or moisture applied immediately prior to using the skin care product. When the activating liquid is other than aqueous, the liquid is either first applied to the skin and the solid film thereafter used or the thin film can be dissolved in the liquid immediately prior to use and the combined product applied to the skin.

Any agents that improve or treat skin when applied topically may be easily and inexpensively added to the thin film composition. These agents may include, for example, nutrients such as vitamins, minerals, amino acids, anti-oxidants, sunscreen agents, or one or more skin peeling compounds (e.g., alpha-hydroxyisobutyric acid). Additionally, one or more preservatives (e.g., isobutylparaben) or fragrances may also be added to the composition.

Examples of other materials having skin improvement activity include allantoin, aloe extract, ginseng extract, placenta extract, bovine blood freed of protein, and fermentation metabolites.

Examples of the materials having the activity to inhibit peroxide lipid are a hydroxy acid, vitamin A, β carotene, vitamin B12, vitamin E, pigments such as dimethylaminostyryl heptyl methyl thiazolium iodide and platonin, scutellaria root, rutin, sesame extract and tea extract. As the cell activators, Quaternium-45, glycolic acid, γ amino acid, sialic acid, royal jelly, and ginseng extract, etc. are known.

Known cell activators also include materials which promote collagen synthesis and materials having the activity to improve the skin. Examples of such materials include ascorbic acid, various growth factors such as transforming growth factor β1, platelet-derived growth factor, fibroblast growth factor and insulin-like growth factor 1, and silk protein.

A viscosity-enhancing agent, such as carboxymethylcellulose or a derivative thereof or polyacrylic acid polymer in a concentration of about 0.1% to about 2% by weight may also be added to the aqueous composition.

Color additives, foundations, moisturizers and skin treatments may be incorporated into rapidly dissolving films for use in applying unit dosed applications of cosmetic colorants, foundations, moisturizers and skin treatments to various parts of the face and body, including, but not limited to, the eyes, lips, hands, and face.

The colorants, foundations, moisturizers and treatments, which may be incorporated into the delivery system, include, but are not limited to iron oxides, mica, alpha hydroxy acids, beta hydroxy acids, TiO2 and mica, polymethyl methacrylate [beads], salicylic acid, and benzoyl peroxide.

The film may be used to deliver the active ingredient or cosmetic by several different techniques depending on the specific film and active or cosmetic ingredient. In a first embodiment, the film is utilized by dissolving with sufficient amounts of water to form a smooth spreadable composition of a slurry or cream like consistency and smoothing the composition onto the user's skin.

In a second embodiment the film is utilized by dissolving with sufficient amounts of a liquid other than water, such as an alcohol or other cosmetically acceptable solvent for the polymer, to form a smooth spreadable composition of slurry or cream-like consistency and smoothing the composition onto the users skin.

In a third embodiment the film formulation is applied to the skin in solid form and is activated by body heat instead of the addition of a liquid. In this embodiment the melt temperature of the polymer is preferably in the range of 70 to 95 0 F.

In other embodiments, the film is striped with different side-by-side formulations, or layered with distinct film formula laminations.

The amount of active ingredient that can be contained in a thin film is limited by a number of constraints, primary among those being film thickness, strip size, solubility, brittleness and curling.

There are a variety of film formers that can be used in films or strips. Generally, these film formulations include the addition of a plasticizer to prevent the film from becoming too brittle and cracking or otherwise degrading during storage and handling.

There are limitations to the amount of the plasticizer that can be used in the film. When excessive plasticizer is employed, the film looses structural integrity, becomes too flimsy and significantly, becomes sticky such that it tends to adhere to other strips of film in the package forming a block. This is especially true when the strips are stacked in a vial, which is a preferred form of primary packaging. In general, from about 0 to about 50 percent plasticizer may be present, preferably form about 0 to about 15 percent. In multi-layer films, the amount of plasticizer present may vary in each film layer.

The disclosed films solve the problems associated with high loadings of active ingredients by laminating multiple layers of thin films to one another, where the films have physical and/or chemical properties which are modified depending on the function that layer plays in the laminate structure. Thus, higher loading may be provided in inner layers with the outer layers having lower loadings or even no actives present. In general the mount of actives that may be utilized ranges from 0 to 70 percent by weight. In most cases it is preferred to limit the amount of actives to less than twenty percent.

This disclosure provides for the use of multiple layers to increase active loading significantly beyond current single or bi-layer films. In general, there may be any number of layers of film in excess of two. For most applications, 3 to 5 layer laminates will provide the expected benefits.

The thickness of the single or multi-layer films can vary widely. It is preferably that the thickness be in the range of from about 0.25 to about 12 mils, preferably from about 0.5 to about 2 mils. It should be recognized that multi-layer films will be thicker than single layer films.

For example, one preferred embodiment contains a bottom layer with high plasticizer content, a middle layer with a high active loading and a top layer with high plasticizer content. The resulting film lamination or sandwich remains flat and flexible even if the middle layer became brittle.

Where multiple active ingredients are desired, each may reside in its own laminate layer having different physical or chemical properties. Where two active ingredients are inter-reactive, the separate layers will significantly extend the shelf life of the product or even allow the development of new products by preventing interaction until utilized by the customer or patient.

A further embodiment anticipates that one or more of these layers may also contain air bubbles that increase surface area for improved dissolution.

These films can be manufactured individually and the multiple layers combined after manufacture but in a preferred embodiment the films are manufactured simultaneously. The simultaneous manufacturing process requires the selection of thin films that have the correct rheology so that they don't merge into one another during the manufacturing process.

Exemplary coating methods are a slot die for up to 3 layers of film slurries that have a viscosity range of approximately 15 Cp to 20,000 Cp, or a slide coater for up to 18 layers of film slurries with a viscosity range of approximately 1 Cp to 500 Cp.

Other coating methods could be used with coating layers that are not coterminious

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

What is claimed is:

1. A method of delivering a cosmetic or skin care product to its site of use comprising
combining the cosmetic or skin care product with a film forming polymer selected from liquid soluble polymers and low-temperature-melt polymers to disperse the skin care product throughout the polymer matrix
drying the mixture into a thin sheet
applying the sheet to the site of use, and
maintaining the sheet in contact with the skin at the site of use for a period of time sufficient for the polymer to melt if it is a low-temperature-melt polymer and release the cosmetic product or to dissolve in a liquid solvent present on the site of use if it is a liquid soluble polymer and release the cosmetic or skin care product.

2. The method of claim 1 where the polymer is a water soluble polymer and moisture present on the users skin is the liquid solvent that dissolves the polymer and releases the cosmetic or skin care product.

3. The method of claim 1 where the polymer is selected from the group consisting of pullulan, hydroxypropylethyl cellulose, hydroxyethyl cellulose, hydroxypropyl cellulose, carboxymethyl cellulose, sodium alginate, pectin, and mixtures thereof.

4. The method of claim 3 where the polymers are selected from the group consisting of pullulan, pectin and mixtures thereof.

5. The method of claim 1 where the polymer is a low temperature-melt polymer and the heat on the users skin melts the polymer and releases the cosmetic or skin care product.

6. The method of claim 5 where the low temperature melt polymer has a melt point in the range of from about 70 to about 95° F.

7. A method of delivering a cosmetic or skin care product to its site of use comprising
a) combining the cosmetic or skin care product with at least one film forming polymer selected from liquid soluble polymers to disperse the skin care product throughout the polymer matrix
b) drying the mixture into a thin sheet,
c) dissolving the liquid soluble polymers in a solvent to release the skin care product, and
d) applying the dissolved cosmetic or skin care product to the site of use.
